(19) United States
(12) Patent Application Publication
(10) Pub. No.: US 2014/0294947 A1
(43) Pub. Date: Oct. 2, 2014

(54) TAMPER RESISTANT IMMEDIATE RELEASE FORMULATIONS

(71) Applicant: PURDUE PHARMA L.P., Stamford, CT (US)

(72) Inventor: Kevin Reilly, Kendall Park, NJ (US)

(21) Appl. No.: 14/344,271

(22) PCT Filed: Sep. 14, 2012
(86) PCT No.: PCT/IB2012/001916

§ 371 Date: Jun. 16, 2014

Related U.S. Application Data
(60) Provisional application No. 61/535,758, filed on Sep. 16, 2011.

(51) Int. Cl. A61K 9/16; A61K 9/48; A61K 31/485

(52) U.S. Cl. CPC A61K 9/1676; A61K 31/485; A61K 9/48
USPC 424/451; 424/490; 514/282

(57) ABSTRACT

Disclosed in certain embodiments is an oral dosage form comprising a plurality of particles, each particle comprising (i) a core comprising a gelling agent; (ii) an optional barrier layer encompassing the core; (iii) an active layer comprising a drug susceptible to abuse encompassing the core or barrier layer; and (iv) an optional controlled release excipient; wherein the dosage form provides an immediate or controlled release; and wherein the viscosity of the dosage form mixed with from about 0.5 to about 10 ml of an aqueous liquid is unsuitable for parenteral or nasal administration.

### FIELD OF THE INVENTION

The present invention relates to the field of pharmaceutical dosage forms that are resistant to tampering and abuse.

### BACKGROUND

Pharmaceutical products are sometimes the subject of abuse. For example, a particular dose of opioid agonist may be more potent when administered parenterally as compared to the same dose administered orally. Some formulations can be tampered with to provide the opioid agonist contained therein for illicit use. Opioid agonist formulations intended for oral use are sometimes crushed or subject to extraction with solvents (e.g., ethanol) by drug abusers to provide the opioid contained therein for non-prescribed illicit use (e.g., nasal or parenteral administration).

There have previously been attempts in the art to control the abuse potential associated with opioid analgesics. For example, the combination of pentazocine and naloxone has been utilized in tablets available in the United States, commercially available as Talwin® Nx from Sanofi-Winthrop. Talwin® Nx contains pentazocine hydrochloride equivalent to 50 mg base and naloxone hydrochloride equivalent to 0.5 mg base. Talwin® Nx is indicated for the relief of moderate to severe pain. The amount of naloxone present in this combination has low activity when taken orally, and minimally interferes with the pharmacologic action of pentazocine. However, this amount of naloxone given parenterally has profound antagonistic action to narcotic analgesics. Thus, the inclusion of naloxone is intended to curb a form of misuse of oral pentazocine which occurs when the dosage form is solubilized and injected. Therefore, this dosage has lower potential for parenteral misuse than previous oral pentazocine formulations. A fixed combination therapy comprising tilidine (50 mg) and naloxone (4 mg) has been available in Germany for the management of severe pain since 1978 (Valoron® N, Goedecke). The rationale for the combination of these drugs is effective pain relief and the prevention of tilidine addiction through naloxone-induced antagonisms at the morphine receptor. A fixed combination of buprenorphine and naloxone was introduced in 1991 in New Zealand (Temgesic® Nx, Reckitt & Colman) for the treatment of pain.

There exists a need in the art for a dosage form containing a drug susceptible to abuse that is resistant to parenteral and/or nasal abuse. In the case of opioid analgesics, there exists a need for a tamper resistant formulation that does not solely rely upon the inclusion of an antagonist in the formulation to deter parenteral and/or nasal abuse.

### SUMMARY OF THE INVENTION

It is an object of certain embodiments of the present invention to provide an immediate release or controlled release solid oral dosage form comprising an active agent (e.g., an opioid analgesic) which is tamper resistant.

It is an object of certain embodiments of the present invention to provide an immediate release or controlled release solid oral dosage form comprising an active agent (e.g., an opioid analgesic) which is subject to less parenteral abuse than other dosage forms.

It is an object of certain embodiments of the present invention to provide an immediate release or controlled release solid oral dosage form comprising an active agent (e.g., an opioid analgesic) which is subject to less intranasal abuse than other dosage forms.

It is a further object of certain embodiments of the present invention to provide an immediate release or controlled release solid oral dosage form comprising an active agent (e.g., an opioid analgesic) which is subject to less diversion than other dosage forms.

It is a further object of certain embodiments of the present invention to treat a disease or condition (e.g., pain) in human patients by administering an immediate release or controlled release solid oral dosage form as disclosed herein to a patient in need thereof

It is a further object of certain embodiments of the present invention to provide a method of treating pain in human patients with an immediate release or controlled release solid oral dosage form comprising an opioid analgesic while reducing the abuse potential of the dosage form.

It is a further object of certain embodiments of the present invention to provide a method of manufacturing an immediate release or controlled release solid oral dosage form of an active agent (e.g., an opioid analgesic) as disclosed herein.

It is a further object of certain embodiments of the present invention to provide a use of a medicament (e.g., an opioid analgesic) in the manufacture of a tamper-resistant dosage form as disclosed herein for the treatment of a disease state (e.g., pain).

The above objects of the present invention and others can be achieved by the present invention, which in certain embodiments is directed to an immediate release solid oral dosage form comprising a plurality of particles, each particle comprising (i) a core comprising a gelling agent; (ii) an optional barrier layer surrounding the core; and (iii) an active layer comprising a drug susceptible to abuse surrounding the barrier layer.

In certain embodiments, the plurality of particles are contained within a pharmaceutically acceptable capsule. In certain other embodiments, the plurality of particles are compressed into a tablet. In certain embodiments, the capsule or tablet further contains a pharmaceutically acceptable diluent.

In certain embodiments, the present invention is directed to a unit dose of an immediate release or controlled release solid oral dosage form, said unit dose comprising from about 2 to about 75 particles, each particle comprising (i) a core comprising a gelling agent; and (ii) an active layer comprising a drug susceptible to abuse surrounding the core. In certain embodiments, the unit dose is contained within a pharmaceutically acceptable capsule. In certain other embodiments, the unit dose is a tablet formed from compression of the particles. In certain embodiments, the unit dose further comprises a pharmaceutically acceptable diluent.

In certain embodiments, the present invention is directed to a unit dose of an immediate release or controlled release solid oral dosage form, said unit dose comprising from about 2 to about 75 particles, each particle comprising (i) a core comprising a gelling agent; (ii) a barrier layer surrounding the core; and (iii) an active layer comprising a drug susceptible to abuse surrounding the barrier layer on the core. In certain embodiments, the unit dose is contained within a pharmaceutically acceptable capsule. In certain other embodiments, the unit dose is a tablet formed from compression of the particles. In certain embodiments, the unit dose further comprises a pharmaceutically acceptable diluent.

In embodiments directed to a controlled release solid oral dosage form, a controlled release excipient can be included in the active layer or can be included in a separate layer surrounding the active layer. In other embodiments, the active layered particles can be incorporated in a controlled release matrix.

In certain embodiments, the solid oral dosage form disclosed herein releases at least about 85% by weight, or at least about 90% by weight, or at least about 95% by weight of the drug within 45 minutes as measured by in-vitro dissolution in a USP Apparatus 1 (basket) at 100 rpm in 900 ml simulated gastric fluid without enzymes (SGF) at 37° C.

In certain embodiments, the solid oral dosage form disclosed herein releases at least about 90% by weight, or at least about 95% by weight, or at least about 98% by weight of the drug within 60 minutes as measured by in-vitro dissolution in a USP Apparatus 1 (basket) at 100 rpm in 900 ml simulated gastric fluid without enzymes (SGF) at 37° C.

In other embodiments, the solid oral dosage form disclosed herein has an in-vitro release rate indicative of controlled release formulation (e.g. for dosing every 12 hours or every 24 hours), as measured by in-vitro dissolution in a USP Apparatus 1 (basket) at 100 rpm in 900 ml simulated gastric fluid without enzymes (SGF) at 37° C. and thereafter switching to 900 ml with Phosphate Buffer at a pH of 7.5 at 37° C.

In other embodiments, the viscosity resulting from mixing a unit dose of the dosage form with from about 0.5 to about 10 ml of an aqueous liquid prevents the drug from being absorbed, or reduces the ability of the drug to be absorbed, by parenteral or nasal administration.

In certain embodiments, the present invention is directed to a process for preparing an immediate release solid oral dosage form comprising a plurality of particles, comprising (i) preparing a plurality of cores, each core comprising a gelling agent; and (ii) applying an active layer comprising a drug susceptible to abuse so that it surrounds each core.

In certain embodiments, the present invention is directed to a process for preparing an immediate release solid oral dosage form comprising a plurality of particles, comprising (i) preparing a plurality of cores, each core comprising a gelling agent; (ii) applying a barrier layer so that it surrounds each core; and (iii) applying an active layer comprising a drug susceptible to abuse so that it surrounds the barrier layer on each core.

In certain embodiments, the present invention is directed to a process for preparing a controlled release solid oral dosage form comprising a plurality of particles, comprising (i) preparing a plurality of cores, each core comprising a gelling agent; and (ii) applying an active layer comprising a drug susceptible to abuse and a controlled release excipient so that it surrounds each core. An optional barrier layer can be between the core and the active layer and/or over the active layer.

In certain embodiments, the present invention is directed to a process for preparing a controlled release solid oral dosage form comprising a plurality of particles, comprising (i) preparing a plurality of cores, each core comprising a gelling agent; (ii) applying an active layer comprising a drug susceptible to abuse so that it surrounds each core and (iii) applying a controlled release layer comprising a controlled release excipient so it surrounds each active layered core. An optional barrier layer can be between the core and the active layer; between the active layer and the controlled release layer and/or over the controlled release layer.

In certain embodiments, the present invention is directed to a process for preparing a controlled release solid oral dosage form comprising a plurality of particles, comprising (i) preparing a plurality of cores, each core comprising a gelling agent; (ii) applying an active layer comprising a drug susceptible to abuse so that it surrounds each core and (iii) dispersing the plurality of active layered cores into a controlled release matrix (e.g., by mixing the cores with a controlled release excipient and compressing into tablets). An optional barrier layer can be between the core and the active layer and/or over the active layer prior to mixing with the excipient.

In certain embodiments, the process further comprises containing the particles within a pharmaceutically acceptable capsule to form a unit dose. In certain other embodiments, the process further comprises compressing the particles into a tablet form to form a unit dose. In certain embodiments, the unit dose comprises from about 2 to about 75 particles.

In certain embodiments, the present invention is directed to a process for the preparation of a dosage form as disclosed herein that releases at least about 85% by weight, or at least about 90% by weight, or at least about 95% by weight of the drug within 45 minutes as measured by in-vitro dissolution in a USP Apparatus 1 (basket) at 100 rpm in 900 ml simulated gastric fluid without enzymes (SGF) at 37° C.

In certain embodiments, the present invention is directed to a process for the preparation of a solid oral dosage form as disclosed herein that releases at least about 90% by weight, or at least about 95% by weight, or at least about 98% by weight of the drug within 60 minutes as measured by in-vitro dissolution in a USP Apparatus 1 (basket) at 100 rpm in 900 ml simulated gastric fluid without enzymes (SGF) at 37° C.

In certain embodiments, the present invention is directed to a process for the preparation of a solid oral dosage form as disclosed herein that releases the active agent indicative of controlled release formulation (e.g. for dosing every 12 hours or every 24 hours), as measured by in-vitro dissolution in a USP Apparatus 1 (basket) at 100 rpm in 900 ml simulated gastric fluid without enzymes (SGF) at 37° C. and thereafter switching to 900 ml with Phosphate Buffer at a pH of 7.5 at 37° C.

In other embodiments, the present invention is directed to a process for the preparation of a dosage form as disclosed herein wherein the viscosity resulting from mixing a unit dose of the dosage form (crushed or uncrushed) with from about 0.5 to about 10 ml of an aqueous liquid prevents the drug from being absorbed, or reduces the ability of the drug to be absorbed, by parenteral or nasal administration.

In certain embodiments, the present invention is directed to a method of treating a disease or condition (e.g., pain, diarrhea or constipation) comprising administering to a patient in need thereof an immediate release dosage form as disclosed herein.

In describing the present invention, the following terms are to be used as indicated below. As used herein, the singular forms “a,” “an,” and “the” include plural references unless the context clearly indicates otherwise. Thus, for example, reference to “an active agent” includes a single active agent as well as a mixture of two or more different active agents, and reference to a “gelling agent” includes a single gelling agent as well as a mixture of two or more different gelling agents, and the like.

As used herein, the terms “active agent,” “active ingredient,” “pharmaceutical agent,” and “drug” refer to any material that is intended to produce a therapeutic, prophylactic, or other intended effect, whether or not approved by a government agency for that purpose. These terms with respect to specific agents include all pharmaceutically active forms of the agent, including the free base form of the agent, and all pharmaceutically acceptable salts, complexes, stereoisomers, crystalline forms, cocrystals, ether, esters, hydrates, solvates, and mixtures thereof, where the form is pharmaceutically active.

As used herein, the terms “therapeutically effective” refers to the amount of drug or the rate of drug administration needed to produce a desired therapeutic result.

As used herein, the terms “prophylactically effective” refers to the amount of drug or the rate of drug administration needed to produce a desired prophylactic result.

As used herein, the term “stereoisomers” is a general term for all isomers of individual molecules that differ only in the orientation of their atoms in space. It includes enantiomers and isomers of compounds with one or more chiral centers that are not mirror images of one another (diastereomers).

The term “enantiomer” or “enantiomeric” refers to a molecule that is non-superimposable on its mirror image and hence optically active wherein the enantiomer rotates the plane of polarized light in one direction by a certain degree, and its mirror image rotates the plane of polarized light by the same degree but in the opposite direction.

The term “chiral center” refers to a carbon atom to which four different groups are attached.

The term “racemic” refers to a mixture of enantiomers.

The term “resolution” refers to the separation or concentration or depletion of one of the two enantiomeric forms of a molecule.

The term “patient” means a subject, particularly a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are entirely normal in all respects or with respect to a particular condition.

“Pharmaceutically acceptable salts” include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like.

The term “ppm” as used herein means “parts per million”. Regarding 14-hydroxycodeinone, “ppm” means parts per million of 14-hydroxycodeinone in a particular sample product. The 14-hydroxycodeinone level can be determined by any method known in the art, preferably by HPLC analysis using UV detection.

### BRIEF DESCRIPTION OF THE DRAWINGS

FIG. 1 is a graphical depiction of the dissolution results of Example 6.

### DETAILED DESCRIPTION

Gelling agents have been contemplated for use in pharmaceutical formulations in order to deter the abuse of dosage forms containing a drug susceptible to abuse (e.g., an opioid analgesic). One form of abuse is the crushing of a controlled release dosage form in order to liberate the drug contained therein for illicit use such as parenteral administration or through absorption across a mucosal surface. When a dosage form having a gelling agent is crushed and then mixed with a solution, a viscosity is obtained which may inhibit the drug from being drawn into a needle, thereby hindering parenteral abuse. Similarly, when the crushed dosage form is applied to a mucosal surface (e.g., the nasal cavity) the composition will gel upon contact with mucosal moisture, thereby inhibiting absorption.

Controlled release dosage forms of drugs of abuse have received considerable attention in an attempt to develop tamper-resistant technologies as the crushing of the dosage form may liberate an amount of active agent normally intended for prolonged release (e.g., 12 to 24 hours)

Immediate release dosage forms are also the subject of abuse and present public safety issues when administered by other than the intended route. One problem to overcome in incorporating a gelling agent into an immediate release dosage form is controlled release characteristics that such an agent may impart to a dosage form when included in sufficient amounts to inhibit tampering.

In certain situations, an immediate release or controlled release dosage form can be abused without crushing, e.g., by contacting the intact dosage form with a liquid to dissolve the active agent contained therein. This can be a particular issue with intact dosage forms that are in particulate form, given the larger surface area and increased dissolution of such dosage forms.

Both controlled release and immediate release multiparticulate formulations may have formulation and pharmacokinetic issues such as (i) difficulty in manufacture, (ii) dose to dose variability in active agent, (iii) pharmacokinetic variability, (iv) variability due to administration with food, and (v) patient-to-patient variability which may be addressed by the present invention.

Immediate release and controlled release dosage forms play a vital role in the management of both acute and chronic conditions (e.g., pain management with opioid analgesics). Therefore, it is important to provide a tamper-resistant dosage form of a drug susceptible to abuse that may be utilized for either controlled or immediate release dosage forms to obtain a viable product that can provide effective plasma levels to a patient according to an intended release profile.

In certain embodiments, the present invention is directed to an immediate release solid oral dosage form comprising a plurality of particles, each particle comprising:
(i) a core comprising a gelling agent; and
(ii) an active layer comprising a drug susceptible to abuse surrounding the core;
wherein the dosage form releases at least about 85% by weight of the drug within 45 minutes as measured by in-vitro dissolution in a USP Apparatus 1 (basket) at 100 rpm in 900 ml simulated gastric fluid without enzymes (SGF) at 37° C.; and wherein the viscosity resulting from mixing a unit dose of the dosage form with from about 0.5 to about 10 ml of an aqueous liquid prevents the drug from being absorbed, or reduces the ability of the drug to be absorbed, by parenteral or nasal administration.

In certain other embodiments, the present invention is directed to an immediate release solid oral dosage form comprising a plurality of particles, each particle comprising:
(i) a core comprising a gelling agent;
(ii) a barrier layer surrounding the core; and
(iii) an active layer comprising a drug susceptible to abuse surrounding the barrier layer on the core;
wherein the dosage form releases at least about 85% by weight of the drug within 45 minutes as measured by in-vitro dissolution in a USP Apparatus 1 (basket) at 100 rpm in 900 ml simulated gastric fluid without enzymes (SGF) at 37° C.; and
wherein the viscosity resulting from mixing a crushed unit dose of the dosage form with from about 0.5 to about 10 ml of an aqueous liquid prevents the drug from being absorbed, or reduces the ability of the drug to be absorbed, by parenteral or nasal administration.

In other embodiments, the present invention is directed to a unit dose of an immediate release oral dosage form comprising from about 2 to about 75 particles, each particle comprising:
(i) a core comprising a gelling agent; and
(ii) an active layer comprising a drug susceptible to abuse surrounding the core;
wherein the dosage form releases at least about 85% by weight of the drug within 45 minutes as measured by in-vitro dissolution in a USP Apparatus 1 (basket) at 100 rpm in 900 ml simulated gastric fluid without enzymes (SGF) at 37° C.; and
wherein the viscosity resulting from mixing a unit dose of the dosage form with from about 0.5 to about 10 ml of an aqueous liquid prevents the drug from being absorbed, or reduces the ability of the drug to be absorbed, by parenteral or nasal administration.

In other embodiments, the present invention is directed to a unit dose of an immediate release oral dosage form comprising from about 2 to about 75 particles, each particle comprising:
(i) a core comprising a gelling agent;
(ii) a barrier layer surrounding the core; and
(iii) an active layer comprising a drug susceptible to abuse surrounding the barrier layer on the core;
wherein the dosage form releases at least about 85% by weight of the drug within 45 minutes as measured by in-vitro dissolution in a USP Apparatus 1 (basket) at 100 rpm in 900 ml simulated gastric fluid without enzymes (SGF) at 37° C.; and
wherein the viscosity resulting from mixing a unit dose of the dosage form with from about 0.5 to about 10 ml of an aqueous liquid prevents the drug from being absorbed, or reduces the ability of the drug to be absorbed, by parenteral or nasal administration.

In alternative embodiments, each particle of the immediate release dosage form may further comprise:
(iv) a second barrier layer encompassing the active layer; and
(v) a second active layer comprising a non-orally bioavailable opioid antagonist encompassing the second barrier layer.

In further embodiments, any of the immediate release dosage forms disclosed herein can be modified to provide a controlled release of the active agent. For example, a controlled release excipient can be included in the active layer or can be included in a separate layer surrounding the active layer. In other embodiments, the active layered particles can be incorporated in a controlled release matrix.

Embodiments with a controlled release excipient (e.g., a release modifying polymer as disclosed herein) can have the excipient coated onto the underlying substrate in a sufficient amount to provide a desired release of the active agent (e.g., a 12 or 24 hour formulation). The active agent can be included in the controlled release layer of the controlled release layer can be incorporated as a separate layer. The controlled release layer can be applied to the underlying substrate in an amount, e.g., to provide a weight fain to the formulation of about 10% to about 30%, or about 15% to about 25%.

In other embodiments, the immediate release formulation disclosed herein can be mixed with a controlled release excipient (e.g., a release modifying polymer as disclosed herein) and subsequently compressed into a tablet. The controlled release would be achieved by erosion of the matrix, diffusion of the active through the matrix, or a combination thereof.

In embodiments with a barrier layer, the material and/or amount of material utilized preferably will not substantially interfere with the release profile of the active agent from the dosage form. The material for the barrier layer can be, e.g., an acrylic polymer, a cellulosic polymer or a vinyl polymer. Preferred barrier layers of the present invention include hydroxypropylmethylcellulose, polyvinyl alcohol, povidone or a mixture thereof.

In other embodiments, the plurality of particles are contained within a pharmaceutically acceptable capsule, preferably with the co-containment of an inert diluent. The diluent can be selected from saccharides (e.g., sucrose, dextrose, lactose, fructose, mannitol, and mixtures thereof), polyethylene glycols and cellulosic materials (e.g., microcrystalline cellulose).

The use of a barrier layer, co-containment with a diluent in a capsule, and selection of the number of particles in a unit dose, may all contribute to reducing agglomeration of the particles upon introduction to a dissolution medium either in vitro or in vivo (e.g., upon oral administration to a human subject) in order to maintain the immediate release profile of the dosage form.

In certain embodiments, the viscosity after mixing a dosage form (crushed or intact) with from about 0.5 to about 10 ml of an aqueous liquid is from about 10 cP to about 100 Cp; from about 25 cP to about 75 Cp; at least about 20 cP; at least about 40 cP or at least about 60 cP. In other embodiments, the viscosity after mixing a dosage form (crushed or intact) with from about 0.5 to about 10 ml of an aqueous liquid is at least about 10 cP, at least about 25 cP, at least about 75 Cp; at least about 100 cP; at least about 150 cP.

In certain embodiments, the weight amount of gelling agent contained in the dosage form of the present invention is not more than the weight amount of drug. In other embodiments, the weight amount of gelling agent contained in the immediate release dosage forms of the present invention is less than the weight amount of drug. In further embodiments, the weight amount of gelling agent contained in the immediate release dosage forms of the present invention is more than the weight amount of drug.

In certain embodiments, the dosage forms of the present invention contain a weight ratio of gelling agent to drug from about 5:1 to about 1:5; from about 3:1 to about 1:3; from about 1:1 to about 1:1.5; from about 1.5:1 to about 1:1; about 1:1.25; or about 1.25:1.

The gelling agent utilized in the dosage forms of the present invention can be selected from sugars, sugar derived alcohols (e.g., mannitol, sorbitol, and the like), starch and starch derivatives, cellulose derivatives (e.g., microcrystalline cellulose, sodium caboxymethyl cellulose, methylcellulose, ethyl cellulose, hydroxyethyl cellulose, hydroxypropyl cellulose, and hydroxypropyl methylcellulose), attapulgites, bentonites, dextrins, alginates, carrageenan, gum tragacanth, gum acacia, guar gum, xanthan gum, pectin, gelatin, kaolin, lecithin, magnesium aluminum silicate, carbomers, carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof. In certain embodiments, the gelling agent is xanthan gum. In other embodiments, the gelling agent is pectin. The pectin or pectic substances include purified or isolated pectates and crude natural pectin from sources such as apple, citrus or sugar beet residues which have been subjected, when necessary, to esterification or de-esterification (e.g., by alkali or enzymes). The pectins may also be derived from citrus fruits such as lime, lemon, grapefruit, and orange. In preferred embodiments, the gelling agent is selected from the group consisting of polyethylene oxide, hydroxypropylcellulose, hydroxyethylcellulose, hydroxypropylmethylcellulose, and mixtures thereof.

A unit dose of a dosage form of the present invention may include without limitation, from about 2 to about 75 particles; from about 10 to about 50 particles; from about 15 to about 25 particles; or from about 10 to about 50 particles. In other embodiments, a unit dose of an immediate release dosage form of the present invention may include without limitation, from about 50 to about 500 particles; from about 75 to about 350 particles; from about 100 to about 300 particles; or from about 150 to about 250 particles. In certain embodiments of the present invention, each unit dose in a batch contains the same amount of particles. Such embodiments may be preferable to typical multiparticulate dosage forms which may contain a greater number of particles and may not uniformly contain the same amount of particulates in each unit dose. Further, typical multiparticulate dosage forms may not have content uniformity for each individual particle.

In certain embodiments, each particle contains a sub-therapeutic amount of active agent but collectively in a unit dosage form contain a therapeutic amount of the active agent.

Certain embodiments of the present invention may address formulation and pharmacokinetic issues such as (i) difficulty in manufacture, (ii) dose to dose variability in active agent, (iii) pharmacokinetic variability, (iv) variability due to administration with food, and (v) patient-to-patient variability.

The particles of the present invention may have a mean diameter from about 0.1 mm to about 10 mm; from about 0.5 mm to about 8 mm; from about 1 mm to about 6 mm; or from about 2 mm to about 4 mm.

The core of the particles of the formulation can be any suitable form such as a powder, a granule, a spheronized bead or a compressed tablet.

The dosage forms of the present invention may comprise without limitation, from about 25% to about 99% core by weight; from about 50% to about 95% core by weight; or from about 65% to about 85% core by weight.

In certain embodiments, the barrier layer is applied to the core in an amount to provide a weight gain from about 1% (w/w) to about 10% (w/w); or from about 4% (w/w) to about 7% (w/w).

In certain embodiments, the immediate release dosage form of the present invention releases at least about 85% by weight, at least about 90% by weight, or at least about 95% by weight of the drug within 45 minutes as measured by in-vitro dissolution in a USP Apparatus 1 (basket) at 100 rpm in 900 ml simulated gastric fluid without enzymes (SGF) at 37° C.

In certain embodiments, the immediate release dosage form of the present invention releases at least about 90% by weight, at least about 95% by weight, or at least about 98% by weight of the drug within 60 minutes as measured by in-vitro dissolution in a USP Apparatus 1 (basket) at 100 rpm in 900 ml simulated gastric fluid without enzymes (SGF) at 37° C.

### Additional Excipients

The compressed cores of the present invention can include additional excipients in order to, e.g., aid manufacturing, provide additional tamper resistance, modify the release rate, or provide alcohol resistance.

The additional excipient may be at least one excipient selected from the group consisting of bulking agents, plasticizers, stabilizers, diluents, lubricants, binders, granulating aids, colorants, flavorants, and glidants.

In certain embodiments, the dosage form includes a polymer that can modify the release rate of the active agent contained therein. Examples of polymers that can be utilized to modify the release of the active agent include pharmaceutically acceptable cellulosic polymers, including but not limited to cellulose esters, cellulose diesters, cellulose triesters, cellulose ethers, cellulose ester-ethers, cellulose acylates, cellulose diacylates, cellulose triacylates, cellulose acetates, cellulose diacetates, cellulose triacetates, cellulose acetate propionates, cellulose acetate butyrates and mixtures thereof. Preferably, the cellulosic polymer is an alkyl cellulosic polymer such as methylcellulose or ethylcellulose.

In other embodiments of the present invention, the release modifying polymer is a pharmaceutically acceptable acrylic polymer selected without limitation from from acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid) (anhydride), methyl methacrylate, polymethacrylate, poly(methyl methacrylate), poly(methyl methacrylate) copolymer, polyacrylamide, aminoalkyl methacrylate copolymer, poly(methacrylic acid anhydride), glycidyl methacrylate copolymers, and mixtures of any of the foregoing.

Preferably, the acrylic polymer is a neutral acrylic polymer (e.g., Eudragit NE 30 D®, Eudragit NE 40 DS or Eudragit NM 30 D®), which can also provide crush-resistant characteristics to the dosage form.

Individual compressed cores can also include a film coating or barrier coating to enhance cosmetic appearance, reduce tackiness and/or provide stability. Examples of materials to be utilized as a film or barrier coat include hydroxypropylmethylcellulose, polyvinyl alcohol, lactose or a mixture thereof. The film or barrier coat can be (i) an coating directly coated onto a compressed core, (ii) an outer coating directly coated onto a final layered compressed core, or (iii) an intermediate layer between any components of the dosage form.

### Controlled Release Formulations

In certain embodiments, dosage forms of the present invention provide a controlled release to provide, e.g., a 6-hour, or an 8-hour, or a 12-hour or a 24-hour formulation.

For 12 hour formulations, the dosage form can, e.g., provide a dissolution release rate in-vitro of the active agent (e.g. an opioid analgesic), when measured by the USP Basket Method at 100 rpm in 700 ml SGF (with or without enzymes) at 37° C. of at least about 15% by weight of the active agent released at 1 hour and thereafter switching to 900 ml with Phosphate Buffer at a pH of 7.5 at 37° C., of from about 25% to about 65% by weight of the active agent released at 2 hours, from about 45% to about 85% by weight of the active agent released at 4 hours, and at least about 60% by weight of the active agent released at 8 hours.

For 24 hour formulations, the dosage form can, e.g., provide a dissolution release rate in-vitro of the active agent (e.g. an opioid analgesic), when measured by the USP Basket Method at 100 rpm in 700 ml SGF (with or without enzymes) at 37° C. for 1 hour and thereafter switching to 900 ml with Phosphate Buffer at a pH of 7.5 at 37° C., of at least about 20% by weight of the active agent released at 4 hours, from about 20% to about 65% by weight of the active agent released at 8 hours, from about 45% to about 85% by weight of the active agent released at 12 hours, and at least about 80% by weight of the active agent released at 24 hours.

In certain embodiments, the amount of active agent (e.g. an opioid analgesic) released at 1 hour is from about 10% (w/w) to about 30% (w/w) as measured by an in-vitro dissolution in a USP Apparatus 1 (basket) at 100 rpm in 900 ml SGF (with or without enzymes) at 37° C.

In certain embodiments, the amount of active agent (e.g. an opioid analgesic) released at 2 hours is from about 25% (w/w) to about 50% (w/w) as measured by an in-vitro dissolution in a USP Apparatus 1 (basket) at 100 rpm in 900 ml SGF (with or without enzymes) at 37° C. with a switch at 1 hour to 900 ml with Phosphate Buffer at a pH of 7.5 at 37° C.

In certain embodiments, the amount of active agent (e.g. an opioid analgesic) released at 4 hours is from about 40% (w/w) to about 80% (w/w) as measured by an in-vitro dissolution in a USP Apparatus 1 (basket) at 100 rpm in 900 ml SGF (with or without enzymes) at 37° C. with a switch at 1 hour to 900 ml with Phosphate Buffer at a pH of 7.5 at 37° C.

In certain embodiments, the amount of active agent (e.g. an opioid analgesic) released at 8 hours is from about 65% (w/w) to about 95% (w/w) as measured by an in-vitro dissolution in a USP Apparatus 1 (basket) at 100 rpm in 900 ml SGF (with or without enzymes) at 37° C. with a switch at 1 hour to 900 ml with Phosphate Buffer at a pH of 7.5 at 37° C.

In certain embodiments, the amount of active agent (e.g. an opioid analgesic) released at 12 hours is greater than about 80% (w/w) as measured by an in-vitro dissolution in a USP Apparatus 1 (basket) at 100 rpm in 900 ml SGF (with or without enzymes) at 37° C. with a switch at 1 hour to 900 ml with Phosphate Buffer at a pH of 7.5 at 37° C.

In certain embodiments, the amount of active agent (e.g. an opioid analgesic) released at 1 hour is from about 15% (w/w) to about 25% (w/w) as measured by an in-vitro dissolution in a USP Apparatus 1 (basket) at 100 rpm in 900 ml SGF (with or without enzymes) at 37° C.

In certain embodiments, the amount of active agent (e.g. an opioid analgesic) released at 2 hours is from about 30% (w/w) to about 40% (w/w) as measured by an in-vitro dissolution in a USP Apparatus 1 (basket) at 100 rpm in 900 ml SGF (with or without enzymes) at 37° C. with a switch at 1 hour to 900 ml with Phosphate Buffer at a pH of 7.5 at 37° C.

In certain embodiments, the amount of active agent (e.g. an opioid analgesic) released at 4 hours is from about 55% (w/w) to about 75% (w/w) as measured by an in-vitro dissolution in a USP Apparatus 1 (basket) at 100 rpm in 900 ml SGF (with or without enzymes) at 37° C. with a switch at 1 hour to 900 ml with Phosphate Buffer at a pH of 7.5 at 37° C.

In certain embodiments, the amount of active agent (e.g. an opioid analgesic) released at 8 hours is from about 75% (w/w) to about 85% (w/w) as measured by an in-vitro dissolution in a USP Apparatus 1 (basket) at 100 rpm in 900 ml SGF (with or without enzymes) at 37° C. with a switch at 1 hour to 900 ml with Phosphate Buffer at a pH of 7.5 at 37° C.

In certain embodiments, the amount of active agent (e.g. an opioid analgesic) released at 12 hours is greater than about 90% (w/w) as measured by an in-vitro dissolution in a USP Apparatus 1 (basket) at 100 rpm in 900 ml SGF (with or without enzymes) at 37° C. with a switch at 1 hour to 900 ml with Phosphate Buffer at a pH of 7.5 at 37° C.

In certain embodiments, the amount of active agent (e.g. an opioid analgesic) released at 1 hour is from about 10% (w/w) to about 30% (w/w); the amount of active agent released at 2 hours is from about 25% (w/w) to about 50% (w/w); the amount of active agent released at 4 hours is from about 40% (w/w) to about 80% (w/w); the amount of active agent released at 8 hours is from about 65% (w/w) to about 95% (w/w), and the amount of active agent released at 12 hours is greater than about 80% (w/w); in each case, as measured by an in-vitro dissolution in a USP Apparatus 1 (basket) at 100 rpm in 900 ml simulated gastric fluid without enzymes (SGF) at 37° C.

In certain embodiments, the amount of active agent released at 1 hour is from about 15% (w/w) to about 25% (w/w); the amount of active agent released at 2 hours is from about 30% (w/w) to about 40% (w/w); the amount of active agent released at 4 hours is from about 55% (w/w) to about 75% (w/w); the amount of active agent released at 8 hours is from about 75% (w/w) to about 85% (w/w), and the amount of active agent released at 12 hours is greater than about 90% (w/w); in each case, as measured by an in-vitro dissolution in a USP Apparatus 1 (basket) at 100 rpm in 900 ml simulated gastric fluid without enzymes (SGF) at 37° C.

### Alcohol Resistance

The gelling agent and the optional release modifying agent can be selected in order to inhibit dose dumping of the active agent in the presence of alcohol. This characteristic is to prevent the dosage form from releasing the active agent at a rate faster than intended when alcohol is imbibed during residence of the dosage form in the gastrointestinal tract. Certain hydrophilic polymers (e.g., polyethylene oxide or methylcellulose) are suitable gelling agents that can provide alcohol resistance to the dosage form.

In certain embodiments, the amount of active agent (e.g. an opioid analgesic) released at 1 hour in 900 mL 0.1 N HCl (pH 1.5) with 40% EtOH using USP Apparatus II at 50 rpm is not more than the amount of active agent released at 1 hour in 900 mL 0.1. N HCl (pH 1.5) with 0% EtOH using USP Apparatus II at 50 rpm.

In certain embodiments, the amount of active agent (e.g. an opioid analgesic) released at 1 hour in 900 mL 0.1 N HCl (pH 1.5) with 40% EtOH using USP Apparatus II at 50 rpm is less than the amount of active agent released at 1 hour in 900 mL 0.1 N HCl (pH 1.5) with 0% EtOH using USP Apparatus II at 50 rpm.

In certain embodiments, the amount of active agent (e.g. an opioid analgesic) released at 1 hour in 900 mL 0.1 N HCl (pH 1.5) with 40% EtOH using USP Apparatus II at 50 rpm is within 25% (w/w) of the amount of active agent released at 1 hour in 900 mL 0.1 N HCl (pH 1.5) with 0% EtOH using USP Apparatus II at 50 rpm.

In certain embodiments, the amount of active agent (e.g. an opioid analgesic) released at 1 hour in 900 mL 0.1 N HCl (pH 1.5) with 40% EtOH using USP Apparatus II at 50 rpm is within 10% (w/w) of the amount of active agent released at 1 hour in 900 mL 0.1 N HCl (pH 1.5) with 0% EtOH using USP Apparatus II at 50 rpm.

In certain embodiments, the amount of active agent (e.g. an opioid analgesic) released at 2 hour in 900 mL 0.1 N HCl (pH 1.5) with 40% EtOH using USP Apparatus II at 50 rpm is not more than the amount of active agent released at 2 hour in 900 mL 0.1 N HCl (pH 1.5) with 0% EtOH using USP Apparatus II at 50 rpm.

In certain embodiments, the amount of active agent (e.g. an opioid analgesic) released at 2 hour in 900 mL 0.1 N HCl (pH 1.5) with 40% EtOH using USP Apparatus II at 50 rpm is less than the amount of active agent released at 2 hour in 900 mL 0.1 N HCl (pH 1.5) with 0% EtOH using USP Apparatus II at 50 rpm.

In certain embodiments, the amount of active agent (e.g. an opioid analgesic) released at 2 hour in 900 mL 0.1 N HCl (pH 1.5) with 40% EtOH using USP Apparatus II at 50 rpm is within 25% (w/w) of the amount of active agent released at 2 hour in 900 mL 0.1 N HCl (pH 1.5) with 0% EtOH using USP Apparatus II at 50 rpm.

In certain embodiments, the amount of active agent (e.g. an opioid analgesic) released at 2 hour in 900 mL 0.1 N HCl (pH 1.5) with 40% EtOH using USP Apparatus II at 50 rpm is within 10% (w/w) of the amount of active agent released at 2 hour in 900 mL 0.1 N HCl (pH 1.5) with 0% EtOH using USP Apparatus II at 50 rpm.

In certain embodiments, the amount of active agent (e.g. an opioid analgesic) released at 4 hour in 900 mL 0.1 N HCl (pH 1.5) with 40% EtOH using USP Apparatus II at 50 rpm is not more than the amount of active agent released at 4 hour in 900 mL 0.1 N HCl (pH 1.5) with 0% EtOH using USP Apparatus II at 50 rpm.

In certain embodiments, the amount of active agent (e.g. an opioid analgesic) released at 4 hour in 900 mL 0.1 N HCl (pH 1.5) with 40% EtOH using USP Apparatus II at 50 rpm is less than the amount of active agent released at 4 hour in 900 mL 0.1 N HCl (pH 1.5) with 0% EtOH using USP Apparatus II at 50 rpm.

In certain embodiments, the amount of active agent (e.g. an opioid analgesic) released at 4 hour in 900 mL 0.1 N HCl (pH 1.5) with 40% EtOH using USP Apparatus II at 50 rpm is within 25% (w/w) of the amount of active agent released at 4 hour in 900 mL 0.1 N HCl (pH 1.5) with 0% EtOH using USP Apparatus II at 50 rpm.

In certain embodiments, the amount of active agent (e.g. an opioid analgesic) released at 4 hour in 900 mL 0.1 N HCl (pH 1.5) with 40% EtOH using USP Apparatus II at 50 rpm is within 10% (w/w) of the amount of active agent released at 4 hour in 900 mL 0.1 N HCl (pH 1.5) with 0% EtOH using USP Apparatus II at 50 rpm.

### Tamper Resistance

In certain embodiments, the solid oral dosage form of the present invention demonstrates the tamper-resistant characteristic of not breaking or shattering when force is applied to it (by, for example, striking it with a hammer). Instead, the solid oral dosage form flattens without breaking or shattering. This characteristic makes it more difficult for the solid oral dosage form to be abused, by snorting the powder of a shattered tablet, chewing a tablet, or injecting a solution prepared from a shattered tablet. The inclusion of polyethylene oxide can provide tamper-resistant properties. The addition of neutral acrylic polymer also provides these properties.

In certain embodiments, the oral solid dosage form can be flattened without breaking, wherein the thickness of the dosage form after flattening corresponds to no more than about 60%, no more than about 50%, no more than about 40%, no more than about 30%, or no more than about 20% of the thickness of the dosage form before flattening.

In certain embodiments, the amount of active agent (e.g. an opioid analgesic) released at 0.5 hour from a flattened dosage form deviates no more than about 20%, no more than about 15%, or no more than about 10% points from a non-flattened dosage form as measured by an in-vitro dissolution in a USP Apparatus 1 (basket) at 100 rpm in 900 ml simulated gastric fluid without enzymes (SGF) at 37° C.

### Active Agents

In certain embodiments, the active agent used in the solid oral dosage form of the present invention is selected from the group consisting of ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha-two-adrenergic agonists, analgesics, anti-pyretics, anti-inflammatory agents, androgens, local and general anesthetics, anti-addictive agents, anti-androgens, anti-arrhythmic agents, anti-asthmatic agents, anti-cholinergic agents, anti-cholinesterase agents, anti-coagulants, anti-diabetic agents, anti-diarrheal agents, anti-diuretic, anti-emetic agents, pro-kinetic agents, anti-epileptic agents, anti-estrogens, anti-fungal agents, anti-hypertensive agents, anti-microbial agents, anti-migraine agents, anti-muscarinic agents, anti-neoplastic agents, anti-parasitic agents, anti-parkinson's agents, anti-platelet agents, anti-progestins, anti-schizophrenia agents, anti-thyroid agents, anti-tussives, anti-viral agents, atypical anti-depressants, azaspirodecanediones, barbiturates, benzodiazepines, benzothiadiazides, beta-adrenergic agonists, beta-adrenergic antagonists, selective beta-one-adrenergic antagonists, selective beta-two-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, contraceptive agents, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, hematopoietic agents, histamines, histamine antagonists, hormones, 5-hydroxytryptamine antagonists, drugs for the treatment of hyperlipoproteinemia, hypnotics, sedatives, immunosupressive agents, laxatives, methylxanthines, moncamine oxidase inhibitors, neuromuscular blocking agents, organic nitrates, opioid agonists, opioid antagonists, pancreatic enzymes, phenothiazines, progestins, prostaglandins, agents for the treatment of psychiatric disorders, retinoids, sodium channel blockers, agents for spasticity and acute muscle spasms, succinimides, testosterones, thioxan thines, thrombolytic agents, thyroid agents, tricyclic antidepressants, inhibitors of tubular transport of organic compounds, drugs affecting uterine motility, vasodilators, vitamins, and mixtures thereof.

In certain embodiments, the active agent is an opioid agonist. In such embodiments, the opioid agonist is selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof. In certain embodiments, the opioid agonist is selected from the group consisting of codeine, fentanyl, hydromorphone, hydrocodone, oxycodone, dihydrocodeine, dihydromorphine, morphine, tramadol, oxymorphone, pharmaceutically acceptable salts thereof, and mixture thereof.

In certain embodiments, the opioid agonist is oxycodone or a pharmaceutically acceptable salt thereof in an amount, e.g., of about 2.5 mg, 5 mg, 7.5 mg or 10 mg.

In certain embodiments of the present invention, wherein the active agent is oxycodone hydrochloride, oxycodone hydrochloride is used having a 14-hydroxycodeinone level of less than about 25 ppm, less than about 15 ppm, less than about 10 ppm, less than about 5 ppm, less than about 2 ppm, less than about 1 ppm, less than about 0.5 ppm or less than about 0.25 ppm.

WO 2005/097801 A1, U.S. Pat. No. 7,129,248 B2 and US 2006/0173029 A1, all of which are hereby incorporated by reference, describe a process for preparing oxycodone hydrochloride having low levels of 14-hydroxycodeinone.

In certain embodiments, the oral solid dosage form of the present invention comprises an active agent that is an opioid antagonist (with or without an opioid agonist). In such embodiments, the opioid antagonist is selected from the group consisting of amiphenazole, naltrexone, methylnaltrexone, naloxone, nalbuphine, nalorphine, nalorphine dinicotinate, nalmefene, nadide, levallorphan, cyclozocine, pharmaceutically acceptable salts thereof and mixtures thereof.

In certain embodiments, the solid oral dosage form of the present invention comprises an active agent that is a non-opioid analgesic. In such embodiments, the non-opioid analgesic is a non-steroidal anti-inflammatory agent selected from the group consisting of aspirin, celecoxib, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, pharmaceutically acceptable salts thereof and mixtures thereof.

In other embodiments, the present invention is directed to the dosage forms disclosed herein utilizing active agents such as benzodiazepines, barbiturates or amphetamines, their antagonists, or combinations thereof.

Benzodiazepines to be used in the present invention may be selected from alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, and pharmaceutically acceptable salts, hydrates, and solvates and mixtures thereof. Benzodiazepine antagonists that can be used in the present invention include, but are not limited to, flumazenil and pharmaceutically acceptable salts, hydrates, and solvates.

Barbiturates to be used in the present invention include, but are not limited to, amobarbital, aprobarbotal, butabarbital, butalbital, methohexital, mephobarbital, metharbital, pentobarbital, phenobarbital, secobarbital and pharmaceutically acceptable salts, hydrates, and solvates mixtures thereof. Barbiturate antagonists that can be used in the present invention include, but are not limited to, amphetamines and pharmaceutically acceptable salts, hydrates, and solvates.

Stimulants to be used in the present invention include, but are not limited to, amphetamines, such as amphetamine, dextroamphetamine resin complex, dextroamphetamine, methamphetamine, methylphenidate and pharmaceutically acceptable salts, hydrates, and solvates and mixtures thereof. Stimulant antagonists that can be used in the present invention include, but are not limited to, benzodiazepines, and pharmaceutically acceptable salts, hydrates, and solvates as described herein.

### Methods of Manufacture

The present invention is also directed to a process for preparing the immediate release oral dosage forms disclosed herein. In certain embodiments, the process comprises:
(i) preparing a plurality of cores, each core comprising a gelling agent; and
(ii) applying an active layer comprising a drug susceptible to abuse so that it surrounds each core;
wherein the dosage form releases at least about 85% of the drug within 45 minutes as measured by in-vitro dissolution in a USP Apparatus 1 (basket) at 100 rpm in 900 ml simulated gastric fluid without enzymes (SGF) at 37° C.;
wherein the viscosity resulting from mixing a crushed unit dose of the dosage form with from about 0.5 to about 10 ml of an aqueous liquid prevents the drug from being absorbed, or reduces the ability of the drug to be absorbed, by parenteral or nasal administration.

In certain other embodiments, the process comprises:
(i) preparing a plurality of cores, each core comprising a gelling agent;
(ii) applying a barrier layer to surround each core; and
(iii) applying an active layer comprising a drug susceptible to abuse so that it surrounds the barrier layer on each core;
wherein the dosage form releases at least about 85% of the drug within 45 minutes as measured by in-vitro dissolution in a USP Apparatus 1 (basket) at 100 rpm in 900 ml simulated gastric fluid without enzymes (SGF) at 37° C.;
wherein the viscosity resulting from mixing a crushed unit dose of the dosage form with from about 0.5 to about 10 ml of an aqueous liquid prevents the drug from being absorbed, or reduces the ability of the drug to be absorbed, by parenteral or nasal administration.

In other embodiments, the process comprises:
(i) compressing (e.g., into a tablet) a plurality of cores comprising a gelling agent;
(ii) optionally applying a barrier layer surrounding each core; and
(iii) applying an active layer comprising a drug susceptible to abuse so that it surrounds the barrier layer;
wherein the dosage form releases at least about 85% of the drug within 45 minutes as measured by in-vitro dissolution in a USP Apparatus 1 (basket) at 100 rpm in 900 ml simulated gastric fluid without enzymes (SGF) at 37° C.;
wherein the viscosity resulting from mixing a crushed unit dose of the dosage form with from about 0.5 to about 10 ml of an aqueous liquid prevents the drug from being absorbed, or reduces the ability of the drug to be absorbed, by parenteral or nasal administration.

In further embodiments, the process comprises preparing an immediate release oral dosage form comprising from about 2 to about 75 particles prepared according to the invention.

The plurality of particles can be further processed into a unit dosage form, e.g., by containment in a capsule; containment in a sachet or paper; or compression into a tablet.

When the particles are contained in a capsule, certain embodiments include the co-containment of a diluent in the capsule along with the active agent particles. The incorporation of the diluent may serve to reduce agglomeration of the particles which can facilitate the dosage form maintaining the desired immediate release profile.

The diluent can be mixed with the particles and the mixture added to the capsule. Alternatively, the diluent can be added to the capsule before or after adding the active agent particles into the capsule.

In a preferred embodiment, the active agent particles are contained in the capsule and back-filled with the diluent.

The diluent can be as disclosed above and is preferably lactose or polyethylene glycol.

### Methods of Treatment

The present invention is further directed to a method of treating a disease or condition comprising administering any of the solid oral dosage forms described herein to a patient in need thereof. In certain embodiments, the patient is treated for pain, diarrhea, or constipation.

The method of treatment of the present invention may comprise administering the solid oral dosage form described herein in combination with another pharmaceutical composition. In certain embodiments, the other pharmaceutical composition is administered to treat the same condition or disease. In other embodiments, the other pharmaceutical composition is administered to treat a different condition or disease.

In certain embodiments, the method of treatment of the present invention further comprises monitoring the patient for how the patient metabolizes the active agent, or how the patient responds to the active agent. In certain embodiments, the method of treatment further comprises altering the dose of the solid oral dosage form in response to said monitoring. In certain embodiments, certain baseline measurements are taken from the patient prior to administering the oral solid dosage form to the patient.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### Example 1

### Preparation of Core Formulation

Cores comprising a gelling agent were prepared in accordance with Table 1:

TABLE 1

| | | Actual Amt Used |
|---|---|---|
| Ingredient | mg/unit | (g) |
| Polyethylene | 3.96 | 1980 |
| Oxide (PEO 303) | | |
| Magnesium | 0.04 | 20 |
| Stearate | | |
| Total | 4.0 | 2000 |

The ingredients were processed according to the following procedure:
1. The polyethylene oxide was added to a polyethylene bag.
2. The magnesium stearate was added and bag blended with the polyethylene oxide for a sufficient time to obtain a substantially uniform blend.
3. The blend was compressed with a Kilian/IMA tablet press with 2 mm multi-tipped tooling with the following parameters:

Target Tablet Weight: 4 mg

Fill Depth/Setting: 3.00-3.05 mm

Main Compression Force Target: 4 Kn

Actual Compression Force: 3.5-4.5 Kn

Actual Tablet Weights: 4.00-4.20 mg

Actual Tablet Thickness: approximately 1.5 mm

### Example 2

### Preparation of Barrier Layered Core Formulation

The cores of Example 1 were coated in accordance with Table 2:

TABLE 2

| | | Actual Amt Used |
|---|---|---|
| Ingredient | mg/unit | (g) |
| Example 1 Cores | 4.00 | |
| Opadry | 0.24 | |
| DI Water* | | |
| Total | 4.24 | |

*DI Water is removed during processing and is not included in final weight

The ingredients were processed according to the following procedure:
1. The DI water was added to a mixer and a vortex was created.
2. The Opadry was added to the DI water and mixed for about 1 hour.
3. The Vector VFC-3 Fluid Bed Processor, 4 liter chamber fitted with a Wurster Column was used to layer the solutions onto the substrate.
4. The processing parameters were as follows: The inlet temperature set point was about 65° to about 70° C.; the product temperature was approximately 45° to about 50° C.; the exhaust temperature was approximately 50° to about 55° C.; the solution spray rate was approximately 4 grams per minute and the process air volume was approximately 70 cfm.
5. The Opadry solution was coated onto the cores of Example 1 to a weight gain of about 6%.

### Example 3

### Preparation of Active Layered Core Formulation

The barrier layered cores of Example 2 were coated in accordance with Table 3:

TABLE 3

| | mg/ | | |
|---|---|---|---|
| | barrier | 20 barrier | |
| | layered | layered | Actual Amt |
| Ingredient | core | cores/unit | Used (g) |
| Barrier Layered | 4.24 | 88.4 | 442 |
| Cores of Example 2 | | | |
| Naltrexone HCl | 0.25 | 5 | 25 |
| Opadry | 0.318 | 6.36 | 31.8 |
| DI Water* | n/a* | n/a* | n/a* |
| Total | 4.988 | 99.76 | 498.8 |

*DI Water is removed during processing and is not included in final weight

1. The DI water was added to a mixer and a vortex was created.
2. The Naltrexone HCl was added to the DI water and mixed until fully dispersed.
3. The Opadry was added to the Naltrexone HCl solution and mixed for about 1 hour.
4. The Vector VFC-3 Fluid Bed Processor, 4 liter chamber fitted with a Wurster Column was used to layer the solutions onto the sub-coated substrate.
5. The processing parameters were as follows: The product temperature set point was about 47° C.; the product temperature was approximately 45° to about 50° C.; the exhaust temperature was approximately 50° to about 55° C.; the solution spray rate was approximately 4 grams per minute and the process air volume was approximately 80 cfm.
6. The Opadry/Naltrexone solution was coated onto the barrier layered cores of Example 2 to a weight gain of about 12.9%.

### Example 4

### Preparation of Seal Layered Core Formulation

The active layered cores of Example 3 were coated in accordance with Table 4:

TABLE 4

| | mg/ | | |
|---|---|---|---|
| | active | 20 active | |
| | layered | layered | Actual Amt |
| Ingredient | core | cores/unit | Used (g) |
| Active Layered | 5.066 | 101.32 | 450 |
| Cores of Ex. 3 | | | |
| Opadry | 0.304 | 6.08 | 27.0 |
| DI Water* | n/a* | n/a* | n/a* |
| Total | 5.370 | 107.4 | 477.0 |

*DI Water is removed during processing and is not included in final weight

1. The DI water was added to a mixer and a vortex was created.
2. The Opadry was added to the DI Water and mixed for about 1 hour.
3. The Vector VFC-3 Fluid Bed Processor, 4 liter chamber fitted with a Wurster Column was used to layer the solutions onto the sub-coated substrate.
4. The processing parameters were as follows: The product temperature set point was about 47° C.; the product temperature was approximately 48° C.; the exhaust temperature was approximately 45° to about 55° C.; the solution spray rate was approximately 4 grams per minute and the process air volume was approximately 70 cfm.
5. The Opadry solution was coated onto the Active layered cores of Example 3 to a weight gain of 6.0%.

### Example 5

### Active Layered Core Formulation Dissolution (Total Amount)

5 mg naltrexone HCl multiple tablet formulations were prepared in accordance with the present invention. The formulations were tested in SGF for the mg amount released at specific time points.

Formulation A was encapsulated with a lactose filler.

Formulation B was encapsulated without a filler.

Formulation C was directly added to the dissolution medium without a capsule.

Formulation D was encapsulated with a polyethylene glycol filler.

The results are set forth in Table 5 below:

TABLE 5

| | Sample wt (mg) | | | | | | | |
|---|---|---|---|---|---|---|---|---|
| | (capsule included) | Vessel | 15.0 min | 30.0 min | 45.0 min | 60.0 min | 120.0 min | 720. [symbol] |
| Formulation A-1 | 259.77 | A | 4.217 | 4.433 | 4.499 | 4.573 | 4.689 | 4. [symbol] |
| Formulation A-1 | 222.45 | A | 4.316 | 4.523 | 4.628 | 4.717 | 4.874 | 4. [symbol] |
| Formulation A-1 | 276.20 | A | 4.425 | 4.666 | 4.768 | 4.834 | 4.976 | 5. [symbol] |
| Mean (n = 3) | | | 4.32 | 4.54 | 4.63 | 4.71 | 4.85 | 4. [symbol] |
| Formulation B-1 | 156.85 | A | 3.756 | 4.080 | 4.260 | 4.403 | 4.737 | 4. [symbol] |
| Formulation B-1 | 160.37 | A | 3.593 | 4.059 | 4.272 | 4.475 | 4.836 | 5. [symbol] |
| Formulation B-1 | 158.99 | A | 3.666 | 4.166 | 4.451 | 4.689 | 5.042 | 5. [symbol] |
| Mean (n = 3) | | | 3.67 | 4.10 | 4.33 | 4.52 | 4.87 | 5. [symbol] |
| Formulation C-1 | 188.56 | B | 4.307 | 4.507 | 4.609 | 4.728 | 4.916 | 4. [symbol] |
| Formulation C-1 | 186.30 | B | 4.376 | 4.542 | 4.626 | 4.708 | 4.870 | 4. [symbol] |
| Formulation C-1 | 187.31 | B | 4.349 | 4.529 | 4.594 | 4.699 | 4.879 | 4. [symbol] |
| Mean | | | 4.34 | 4.53 | 4.61 | 4.71 | 4.89 | 4. [symbol] |
| Formulation D-1 | 402.02 | B | 4.021 | 4.478 | 4.628 | 4.733 | 4.885 | 4.9 |
| Formulation D-1 | 467.58 | B | 4.164 | 4.472 | 4.602 | * | * | 4.7 |
| Formulation D-1 | 425.72 | B | 4.374 | 4.669 | 4.824 | 4.879 | 4.974 | 4.9 |
| Mean (n = 3) | | | 4.19 | 4.54 | 4.68 | 4.81 | 4.93 | 4. [symbol] |

*Samples were not pulled for these time points for vessel #5 of Bath B. [symbol]
indicates data missing or illegible when filed

### Example 6

### Active Layered Core Formulation Dissolution (% Released)

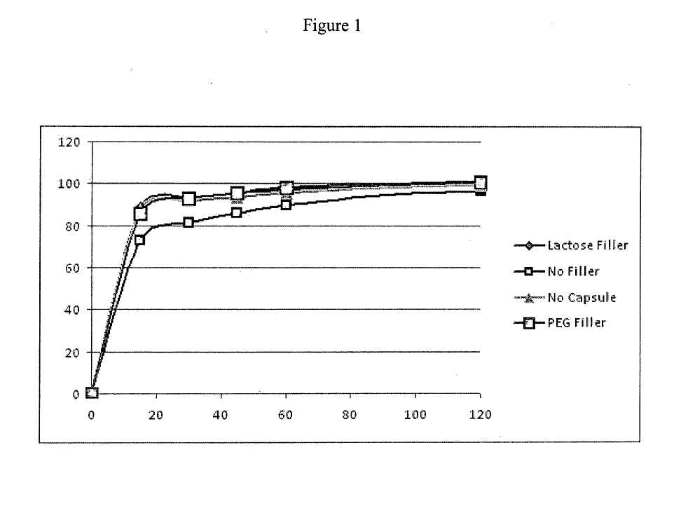

Formulations A-D were tested in SGF for the total amount of naltrexone HCl released over time. The results are set forth in FIG. 1 and Table 6 below:

TABLE 6

Filler Type

| | 0 | 15 | 30 | 45 | 60 | 120 |
|---|---|---|---|---|---|---|
| Formulation A | 0 | 89 | 93 | 95 | 97 | 100 |
| Formulation B | 0 | 73 | 81 | 86 | 90 | 97 |
| Formulation C | 0 | 88 | 92 | 93 | 95 | 99 |
| Formulation D | 0 | 86 | 93 | 96 | 98 | 101 |

### Example 7

### Syringability of Active Layered Core Formulation

A. Materials

Scintillation vial (hand shake for 1-20 min)

1 cc Insulin syringe 28 1/2 gauge needle
B. One active layered core of Example 3 was placed in 1 mL of water. The syringability was tested after specified time periods. The results are set forth in Table 7A below:

TABLE 7A

| Time | | Syringability |
|---|---|---|
| 1~5 | min | slow to pull up |
| 10 | min | slightly viscous, still syringable |
| 20 | min | more viscous, still syringable |

C. 20 active layered cores of Example 3 were placed in 5 mL of water. The syringability was tested after specified time periods. The results are set forth in Table 7B below:

TABLE 7B

| Time | Syringability |
|---|---|
| 1 min | slow to draw up |
| 3 min | same as 1 min |
| 5 min | foamy, difficult to syringe |
| 10 min | very difficult to pull up |

### Example 8

### Syringability of Multiple Active Layered Core Formulations

Multiple tablet formulations prepared in accordance with the present invention were crushed, diluted with 2 ml of water and heated. Variations were as follows:

Formulation 8A was 1 dose (20 tablets) crushed between 2 tablespoons, diluted with 2 ml of water, and heated for 2 minutes (while stirring with a needle) with a butane lighter.

Formulation 8B was a double dose (40 tablets) crushed with a mortar and pestle, transferred to a spoon and heated for 2 minutes (while stirring with a needle) with a butane lighter.

Formulation 8C was a double dose crushed with a mortar and pestle, transferred to a spoon and heated for 2 minutes dry. 2 ml of water was then added and the mixture was stirred with a needle.

Formulations 8D to 8H were all performed using 40 tablets placed in 20 ml scintillation vials. 2 ml of water was added to each and then they each were shaken for 1, 3, 5, 12 & 15 minutes.

Formulation 8D was shaken by hand. Formulations 8E to 8G were mixed on a vortex. Formulation 8H was placed on a wrist action shaker. The liquid was removed using a 1 ml insulin syringe and then placed in a 25 ml volumetric flask and filled with SGF.

Results are set forth in Table 8 below:

TABLE 8

| | | Assay | | |
|---|---|---|---|---|
| | Volume | results in | % of | |
| Formulation | drawn in ml | mg of drug | theoretical | Appearance & observations |
| 8A | 0.8 | 1.7 | 34.2 | Difficult to crush using 2 spoons. |
| | | | | Took 2 minutes to draw up. |
| 8B | 0.15 | 0.6 | 6.4 | Purple in color, much more |
| | | | | viscous. Complete glob after 3 |
| | | | | minutes. Unable to effectively |
| | | | | draw up. |
| 8C | 0.7 | 2.6 | 25.8 | Thin, but not easily syringed. |
| | | | | Cotton used after a few minutes. |
| | | | | Took 6 minutes to draw up. |
| 8D | 2 | 8.3 | 82.8 | Liquid was grayish pink, easily |
| | | | | drawn up. |
| 8E | 1.5 | 7.3 | 72.7 | Liquid was grayish pink, slight |
| | | | | issue with tablets drawn to |
| | | | | needle. |
| 8F | 1.35 | 6.5 | 65.3 | Liquid was grayish pink, more |
| | | | | difficult to avoid tablets on tip of |
| | | | | needle. |
| 8G | 1.2 | 5.2 | 52.3 | Liquid was grayish pink, tablets |
| | | | | were blocking syringe, they were |
| | | | | removed from liquid at top of |
| | | | | vial. Difficulty drawing up. |
| 8H | 1.4 | 5.8 | 58.4 | Liquid was grayish pink. Similar |
| | | | | to 8G. |

### Example 9

### Preparation of Controlled Release Formulation (Prophetic)

The active layered cores of Example 3 are coated with a controlled release coating as follows:
1. An aqueous dispersion of a neutral copolymer based on ethyl acrylate and methyl methacrylate (Eudragit NE 40 D) is coated onto the substrate with a Vector VFC-3 Fluid Bed Processor, 4 liter chamber fitted with a Wurster Column.
2. The processing parameters are as follows: The product temperature set point is set at ambient temperature; the product temperature is ambient temperature; the exhaust temperature is approximately 45° to about 55° C.; the spray rate is approximately 4 grams per minute and the process air volume is approximately 70 cfm.
3. The Eudragit NE 40 D dispersion is coated onto the Active layered cores of Example 3 to a weight gain of 20%.

### Example 10

### Preparation of Controlled Release Formulation (Prophetic)

The active layered cores of Example 3 are coated with a controlled release coating as follows:
1. An aqueous dispersion of a ethylcellulose (Surelease) is coated onto the substrate with a Vector VFC-3 Fluid Bed Processor, 4 liter chamber fitted with a Wurster Column.
2. The processing parameters are as follows: The product temperature set point is set at ambient temperature; the product temperature is ambient temperature; the exhaust temperature is approximately 45° to about 55° C.; the spray rate is approximately 4 grams per minute and the process air volume is approximately 70 cfm.
3. The Eudragit NE 40 D dispersion is coated onto the Active layered cores of Example 3 to a weight gain of 20%.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

1. An oral dosage form comprising a plurality of particles, each particle comprising:
(i) a core comprising a gelling agent;
(ii) a barrier layer encompassing the core; and
(iii) an active layer comprising a drug susceptible to abuse encompassing the barrier layer;
wherein the dosage form releases at least about 85% of the drug within 45 minutes as measured by in-vitro dissolution in a USP Apparatus 1 (basket) at 100 rpm in 900 ml simulated gastric fluid without enzymes (SGF) at 37° C.;
wherein the viscosity of the dosage form mixed with from about 0.5 to about 10 ml of an aqueous liquid is at least about 10 cP.

2. An oral dosage form comprising from about 2 to about 75 particles, each particle comprising:
(i) a core comprising a gelling agent;
(ii) an active layer comprising a drug susceptible to abuse encompassing the core;
wherein the dosage form releases at least 85% of the drug within 45 minutes as measured by in-vitro dissolution in a USP Apparatus 1 (basket) at 100 rpm in 900 ml simulated gastric fluid without enzymes (SGF) at 37° C.; and
wherein the viscosity of the dosage form mixed with from about 0.5 to about 10 ml of an aqueous liquid is at least about 10 cP.

3. An oral dosage form comprising a plurality of particles, each particle comprising:
(i) a compressed core comprising a gelling agent;
(ii) an active layer comprising a drug susceptible to abuse encompassing the core, and;
(iii) a controlled release excipient included in the active layer or layered on the active layer;
wherein the dosage form releases from about 10% to about 30% of the drug at 1 hour as measured by in-vitro dissolution in a USP Apparatus 1 (basket) at 100 rpm in 900 ml simulated gastric fluid without enzymes (SGF) at 37° C.; and
wherein the viscosity of the dosage form mixed with from about 0.5 to about 10 ml of an aqueous liquid is at least about 10 cP.

4. An oral dosage form comprising a plurality of particles, each particle comprising:
(i) a compressed core comprising a gelling agent
(ii) an active layer comprising a drug susceptible to abuse encompassing the core;
wherein the active layered particles are dispersed in a controlled release matrix;
wherein the dosage form releases from about 10% to about 30% of the drug at 1 hour as measured by in-vitro dissolution in a USP Apparatus 1 (basket) at 100 rpm in 900 ml simulated gastric fluid without enzymes (SGF) at 37° C., and
wherein the viscosity of the dosage form mixed with from about 0.5 to about 10 ml of an aqueous liquid is at least about 10 cP.

5. The oral dosage form of claim 1, wherein the viscosity of a crushed dosage form mixed with from about 0.5 to about 10 ml of an aqueous liquid is at least about 10 cP.

6-10. (canceled)

11. The dosage form of claim 1, wherein the ratio of gelling agent to drug is from about 5:1 to about 1:5.

12-16. (canceled)

17. The oral dosage form of claim 1, wherein the gelling agent is selected from the group consisting of sugars, sugar derived alcohols, cellulose derivatives, gums, polymers, and mixtures thereof.

18. The oral dosage form of claim 1, wherein the gelling agent is selected from the group consisting of polyethylene oxide, hydroxypropylcellulose, hydroxyethylcellulose, hydroxypropylmethylcellulose, and mixtures thereof.

19. The dosage form of claim 1, wherein the barrier layer comprises hydroxypropylmethylcellulose, polyvinyl alcohol, povidone or a mixture thereof.

20. The dosage form of claim 1, wherein the plurality of particles comprise a therapeutically effective amount of the drug.

21. The dosage form of claim 1, wherein the drug is selected from the group consisting of an opioid agonist, a tranquilizer, a CNS depressant, a CNS stimulant, a sedative hypnotic, and mixtures thereof.

22. The dosage form of claim 1, wherein the drug is an opioid agonist.

23. The dosage form of claim 22, wherein the opioid agonist is selected from the group consisting of codeine, morphine, oxycodone, oxymorphone, hydrocodone, hydromorphone, pharmaceutically acceptable salts thereof, and mixtures thereof.

24. The dosage form of claim 22, wherein the opioid agonist is oxycodone or a pharmaceutically acceptable salt thereof.

25. The dosage form of claim 24, comprising about 5 mg oxycodone or a pharmaceutically acceptable salt thereof.

26. The dosage form of claim 1, wherein the cores are in the form of compressed tablets.

27. The dosage form of claim 26, comprising from about 2 to about 75 particles.

28-30. (canceled)

31. The dosage form of claim 1, wherein the cores are in the form of speronized beads.

32. The dosage form of claim 1, comprising from about 50 to about 500 particles.

33-35. (canceled)

36. The dosage form of claim 1, comprising from about 25% to about 99% core.

37-38. (canceled)

39. The dosage form of claim 1, wherein the barrier layer is applied to the core in an amount to provide a weight gain from about 1% (w/w) to about 10% (w/w).

40-41. (canceled)

42. The dosage form of claim 1, wherein each particle further comprises:
(iv) a second barrier layer encompassing the active layer; and
(v) a second active layer comprising non-orally bioavailable opioid antagonist encompassing the second barrier layer.

43. The dosage form of claim 1, wherein the plurality of particles are contained within a pharmaceutically acceptable capsule.

44. The dosage form of claim 43, further comprising a diluent contained within the pharmaceutically acceptable capsule.

45-48. (canceled)

49. The dosage form of claim 1, wherein the dosage form releases at least about 90% of the drug within 45 minutes as measured by in-vitro dissolution in a USP Apparatus 1 (basket) at 100 rpm in 900 ml simulated gastric fluid without enzymes (SGF) at 37° C.

50-53. (canceled)

54. A process for preparing an immediate release oral dosage form comprising a plurality of particles, comprising:
(i) preparing a plurality of cores comprising a gelling agent;
(ii) applying a barrier layer encompassing the cores; and
(iii) applying an active layer comprising a drug susceptible to abuse encompassing the barrier layer;
wherein the dosage form releases at least about 85% of the drug within 45 minutes as measured by in-vitro dissolution in a USP Apparatus 1 (basket) at 100 rpm in 900 ml simulated gastric fluid without enzymes (SGF) at 37° C.;
wherein the viscosity of the dosage form mixed with from about 0.5 to about 10 ml of an aqueous liquid is at least about 10 cP.

55-68. (canceled)

69. A method of treating a disease or condition comprising administering to a patent in need thereof, a dosage form according to claim 1.

70. A method of treating pain comprising administering to a patent in need thereof, a dosage form according to claim 22.
